(19) United States
(12) Patent Application Publication
(10) Pub. No.: US 2011/0054305 A1
(43) Pub. Date: Mar. 3, 2011

(54) PHOTON UPCONVERTING IMPLANTABLE MEDICAL DEVICES

(76) Inventor: Stephen Eliot Zweig, Los Gatos, CA (US)

(21) Appl. No.: 12/871,899

(22) Filed: Aug. 30, 2010

Related U.S. Application Data
(60) Provisional application No. 61/242,025, filed on Sep. 14, 2009.
(60) Provisional application No. 61/238,201, filed on Aug. 30, 2009.

(51) Int. Cl. A61B 5/05

(52) U.S. Cl. 600/424

(57) ABSTRACT

Implantable medical devices, such as catheters and stents, which contain light upconverting materials as part of their construction, are disclosed. Such upconverting materials, often rare earth compounds, can convert longer wavelength light, such as light from the 600-800 nm region, which penetrates the body tissue easily, but which is too low energy to induce many chemical reactions, into shorter wavelength light, such as light above 500 nm, which penetrates only a small distance through the body tissues, but which contains enough energy to induce a greater variety of chemical reactions, including conversion of drugs such as psorlens from an inactive to an active form, activation of cytotoxic or antimicrobial photocatalysts such as TiO₂, and conversion of non-target molecules to target molecules for other drug and biotherapeutic systems. Such implantable devices can potentially exert their therapeutic effects for years after administration.

### CROSS REFERENCE TO RELATED APPLICATIONS

This invention claims the priority benefit of provisional patent application No. 61/238,201, “Photon upconverting implantable medical devices”, filed Aug. 30, 2009, and provisional patent application No. 61/242,025, filed Sep. 14, 2009, both invented by Stephen Eliot Zweig.

### BACKGROUND OF THE INVENTION

1. Field of the Invention

The invention covers certain methods and devices designed to improve the performance of implanted medical devices. The invention also covers certain improvements in the field of drug administration technology and drug administration devices. The invention also covers certain improvements in the field of radiation and light administration therapy.

2. Description of the Related Art

Stents, catheters, and other related medical devices have proven useful for the treatment and prevention of preventing coronary artery disease, and other disorders.

Stents are commonly introduced to the inside of a diseased artery or other body lumen using a catheter. The stent is normally inflated inside the artery or body lumen, and the catheter removed. The stent then acts to hold the artery or body lumen open, and can also help prevent subsequent reclosure (stenosis).

Catheters are used for a wide variety of medical purposes. Some purposes, such as cardiac treatment, require only very short-term use in the body. Other purposes, such as administering blood, drugs, and nutrition intravenously, as well as draining urine from the urethra, require that the catheters be implanted into the body for extended periods of time.

One common problem with stents is unwanted cell proliferation and hyperplasia. Often the inner wall of the artery is damaged, either by the disease, or alternatively by irritation induced during the stent introduction or stent material itself. As a result, the body's natural defense mechanisms act to cover the stent material by a process of cell proliferation. If unchecked, the resulting hyperplasia and cell overgrowth in the stent area can act to narrow or close the stent, resulting in blocked circulation, and rendering the stent ineffective.

Numerous prior art attempts have been made to prevent stent closure. These methods include making stents of radioactive material, and embedding various types of cell proliferation inhibiting or cytotoxic drugs into the stent material itself. Radioactive stents have presented safety problems, however, and have not been totally effective. Similarly drug eluting stents, although promising, have not been totally effective either. Stents are small, and only a small amount of drug can be embedded or associated with the stent. Eventually this small amount of drug runs out, and thus the stent no longer can prevent unwanted cell proliferation.

Bernstein, in US patent application 2009/0130169, proposed employing phosophors that converted electromagnetic radiation to cytotoxic ultraviolet UVC radiation (e.g. the 250 to 260 nm range) into stents, and using the damaging effect of UVC radiation on proliferating cells to inhibit hyperplasia. However the efficiency of upconverting light to such short wavelengths will generally be low with most known materials. Additional problems are that these short wavelengths are intensely absorbed by nucleic acids and other materials in the body, and thus their penetration range will be extremely short. Finally, the direct killing power of UVC light is not overly high, and thus large amounts of UVC light will need to be produced in order to produce the desired cytotoxic effect.

Thus methods to improve stent performance, particularly to prevent unwanted cell proliferation, stent closure and restenosis, are desirable.

For catheters, a different type of problem, infection, presents itself. Many catheters span the junction between the non-sterile environment outside the body, and the ideally sterile environment inside the body, and the catheter-body junction presents a location where bacteria, fungi, and other pathogens may enter the body.

Previous workers, such as Yao et. al., “Self-Sterilization using Silicone Catheters Coated with Ag and TiO2 Nanocomposite thin film”, Journal of Biomedical Materials Research Part B: Applied Biomaterials Volume 85B Issue 2, Pages 453-460 (2007) attempted to use the known sterilization capabilities of Titanium dioxide (TiO₂) photocatalysis, which is activated by exposure to UV light, to reduce the danger caused by bacterial colonization of such catheters. However, as they discuss, “it is impossible for the photocatalyst to work in dark conditions such as inside the human body” (p 454 column 1, first paragraph).

Thus methods to generally improve the on-demand cytotoxic or antimicrobial performance of a range of various types of implanted medical materials are also desirable.

### BRIEF SUMMARY OF THE INVENTION

In one embodiment, the invention is a device, system, and method of preventing occlusion of arteries and veins by means of a stent that contains a photon (light) upconverting material. For the purposes of this disclosure, “upconverting material” is a material that is capable of absorbing multiple lower energy (longer wavelength) photons, upconverting these lower energy photons to higher energy photons, and then emitting these higher energy (shorter wavelength) photons.

In another embodiment, the invention is an improved device, system, and method for preventing infection in the body, by means of an implanted medical device, such as a catheter, that contains both an upconverting material and a light activated photocatalyst, such as TiO2 or other material that has antimicrobial or cytotoxic properties upon exposure to light.

In other aspects of the invention include other types of improved implantable medical devices incorporating upconverting material. In addition to the benefits of implantable stents that incorporate upconverting material, a number of other implantable devices, including, but not limited to orthopedic medical implants, spinal implants, brachytherapy seeds or pellets, and drug administration implants may also benefit from this approach.

In yet another aspect of the invention, the specificity of drug administration devices may be further enhanced by strategic use of medical implants constructed using at least some upconverting materials.

The general principle of the invention is to utilize the ability of an upconverting material to convert lower energy (longer wavelength) illuminating photons into higher energy (shorter wavelength) photons that are subsequently emitted by the upconverting material.

Here, the advantages of upconverting materials can be enhanced by taking advantage of the fact that the dominant color absorbing material in the human body is hemoglobin (predominantly oxyhemoglobin). Hemoglobin has very little light absorption in the wavelengths below about 600 nm. Thus, to a first approximation, the human body may be viewed as being semi-transparent or semi-translucent at longer wavelengths, typically considered to be in the near infrared (near IR) region. As a result, such longer wavelengths can travel for a considerable distance in the human body, such that illuminating light sources can be placed some distance away from the illumination target (indeed the light sources may even be placed outside the body in some cases), and the illumination light can travel a significant distance through the human body to reach the upconverting material.

By contrast, the higher energy photons emitted by the upconverting material will often be at a wavelength that is highly absorbed by hemoglobin, and thus the higher energy photons will travel only a very short distance. Because higher energy photons are generally more effective at stimulating a wide variety of different chemical reactions, this combination of short distance (localization) and higher ability to induce chemical reactions can be utilized for a wide variety of therapeutic applications.

In particular, there are a number of upconverting materials that can take illumination light at wavelengths greater than about 600 nm, and in turn upconvert the light and produce either shorter wavelength blue light at wavelengths of around 475 nm or shorter, violet light at wavelengths of 400 or shorter, or ultraviolet light at wavelengths between 400 nm and 320 nm. Even shorter wavelength violet light, Ultraviolet B light, in the 320-280 nm region has been reported.

As previously discussed, the shorter wavelength light produced by the upconverting material is more energetic than the longer wavelength light used to excite the upconverting material, and as a result, the shorter wavelength light may be employed to do a number of useful chemical reactions. In one use, the shorter wavelength light may be used to kill or damage nearby cells directly.

In an alternative use, the shorter wavelength light may be used to activate a light activated cytotoxic drug, such as a psorlen, or a light activated photocatalyst, such as TiO₂, and exhibit desired cytotoxic or antimicrobial effects indirectly.

In yet another alternative use, the shorter wavelength light may be used to convert a non-target molecule to a target-molecule, and the target molecule in turn may be recognized by various receptor molecules, which in turn may deliver various payloads, such as cytotoxic agents, immunological agents, regulatory agents, and the like to cells and tissues nearby the implanted upconverting medical device. In yet another alternative use, the localized and shorter wavelength light may be used to enhance the efficiency of various diagnostic procedures and methods.

Thus by suitable use of upconverting implanted medical devices, longer wavelength incident illumination, and one or more optional photocatalysts, light sensitive drugs and drug combinations, the property of the tissue surrounding the implant may be modified in useful ways.

In the case of a stent, unwanted cell hyperplasia and other proliferation that puts the stent at risk for premature closing can be eliminated. For an orthopedic implant, a suitable immune cell suppressant function might be employed to reduce the amount of inflammation or arthritis in the region. For an implant designed to combat a tumor or unwanted growth, such as the various “seeds” or “pellets” used in brachytherapy to treat diseases such as prostate hyperplasia or cancer, such methods can help direct cytotoxic and inflammatory reducing agents to desired portions of the body.

For an indwelling catheter, risk of infection can be produced by constructing or treating the catheter so that it has both upconverting material and photocatalytic material on or near its surface. Exposure of the catheter to longer wavelength and deeply tissue penetrating light and infrared light, for example light in the 600-800 nm regions, causes the upconverting material to produce shorter wavelength light. This shorter wavelength light activates the photocatalyst (such as TiO₂). The photocatalyst in turn exhibits anti-microbial activities and reduces the amount of bacteria, fungi, and other microorganisms near the surface of the catheter).

### BRIEF DESCRIPTION OF THE DRAWINGS

FIG. 1 shows a stent configuration useful for holding light upconverting particles, and pressing these light upconverting particles against the inner wall of an artery or vein.

FIG. 2 shows a detail of a light upconverting particle or disk.

FIG. 3 shows the light absorption spectra of oxyhemoglobin in the visible and near infrared regions.

FIG. 4 shows examples of various ways to illuminate light upconverting implantable devices such as stents.

FIG. 5 shows how a pellet containing upconverting material may be used, in conjunction with light activated drugs, to treat a tumor or other diseased portion of tissue.

FIG. 6 shows use of small (millimeter size) or microscopic (size of 1000 microns or less) soluble upconverting particles for diagnosis or treatment.

FIG. 7 shows a medical catheter, such as a Broviac® catheter, or other catheter, comprised of both upconverting particles and particles of a photocatalytic antibacterial material such as particles of titanium dioxide, and surrounded by a biofilm of an undesired pathogenic bacteria. By illuminating the catheter with 600-800 nm light, which penetrates deeply into the body, the upconverting particles produce shorter wavelength light, which activates the photoactivated antibacterial material. The photoactivated antibacterial material in turn kills the various undesired pathogenic bacteria in the biofilm.

FIG. 8 shows one possible mechanism by which a particle of an upconverting material can convert infrared light to shorter wavelength light, activating a photocatalyst, and resulting in destruction of microbes.

### DETAILED DESCRIPTION OF THE INVENTION

Throughout this disclosure, stent and catheters will be used as specific examples of implantable medical devices that can be improved by the use of upconverting material. However it should be understood that these examples are simply provided for ease of conveying the basic concept, and are not intended to be limiting in any way.

### Stent Applications:

Some modern stents, such as the stent design of Stanley (U.S. Pat. Nos. 6,565,065 and 7,169,179) (Conor Medsystems) are formed from a deformable material, such as Nitinol (nickel-titanium), other metal or alloy, or other deformable material, and often containing a plurality of small holes. The prior art for such small holes, exemplified by U.S. Pat. No. 7,169,179 is to fill the small holes with therapeutic agents (drugs), often packaged with time release barriers and the like. These therapeutic drugs can then be used to prevent restenosis, thrombosis, or other subsequent unwanted biological response that could diminish the effectiveness of the stent. The drawback of this time release drug concept, however is that the volume of the plurality of small holes is very small, and eventually most or all of the drug will elute.

By contrast, if the small holes are instead filled with particles of an upconverting material, which can often be packaged in the form of a relatively biologically inert glass, then the ability of the upconverting material to convert longer wavelength incident light to shorter wavelength emitted light is effectively infinite. Neglecting upconverting material deterioration, as long as there is a source of illumination light, the upconverting material will produce shorter wavelength photons. These shorter wavelength photons can be produced for many years, if necessary, in contrast to drugs, which often become exhausted within weeks or months.

Further, if the shorter wavelength photons are used in conjunction with appropriate light sensitive therapeutic drugs or other useful photosensitized agents (for example photosensitized agents such as titanium dioxide (TiO₂), cells in the vicinity of the implanted stent can be modified in a variety of ways. These cells can be damaged or destroyed by light triggered cytotoxic drugs, be inhibited by appropriate light triggered target molecules, or be induced to enter other desired therapeutic states depending upon the treatment modality desired. Indeed, a stent or other therapeutic device could be implanted, treated in conjunction with a first available drug (such as a psorlen) or photosensitized agent for a number of years, and then used in conjunction with a new light sensitive drug or photosensitized agent, years later, that may not have even been available at the time that the upconverting stent was originally implanted.

If the stent is constructed with a mixture of upconverting material and photoactivated catalysts (again using TiO2 as a specific example), the photoactivated catalyst may exert its local cytotoxic effect by, for example, producing strong oxidants, such as hydroxyl radicals (OH) and superoxide radicals (O2). Thus, for example, when the upconverting material is exposed to deeply tissue penetrating infrared light, such as light in the 600-800 nm region, the upconverting material produces shorter wavelength light that activates the photocatalyst. The photoactivated catalyst produces cytotoxic agents, such as strongly oxidizing radicals, which in turn can destroy unwanted cell hyperplasia and other proliferation causing unwanted restenosis of the stent.

Further, such methods can also be used for diagnostic purposes. One way is by injecting a suitable dye into circulation. If an upconverting stent is freely exposed to the blood circulation, the shorter wavelength light emitted by the upconverting stent should be able to travel the short distance to an injected dye, and cause the dye to fluoresce or emit some other signal at a third wavelength, that can then be detected. However if the stent is overgrown, the ability of the shorter wavelength emitted light to travel the short distance to the injected dye will be diminished due to the interfering effect of the overgrowing cells, and this effect might also be detected.

Returning to the upconverting stent embodiment, as one example, the upconverting stent can be produced by filling the plurality of holes from a Stanley (U.S. Pat. Nos. 6,565,065/7,169,179) stent design with an upconverting plastic, glass, or ceramic material. The resulting stent would essentially retain its normal characteristics of being capable of being collapsed, employed by a suitable catheter system, and then expanded to fill the appropriate artery or vein.

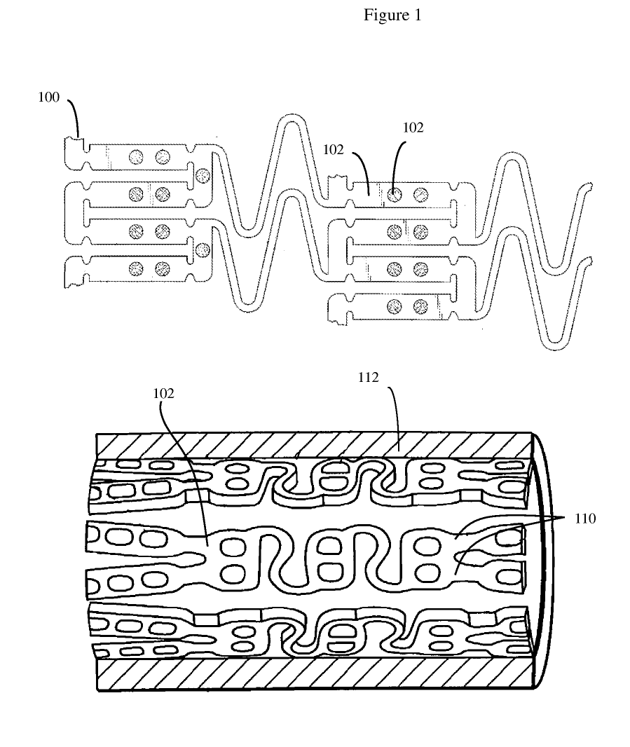

This configuration is shown in FIG. 1. FIG. 1 (100) shows the stent in a flattened configuration, with a plurality of holes (102) in the (usually metal) support material. Here, these holes will be filled with the previously discussed upconverting material, which often will be in the form of small plastic, glass, or ceramic disks, squares, or other shaped particles. In an alternative configuration, the stent surface may be covered a polymer, and small granules of the upconverting material may be embedded in the polymer. In this alternative configuration (not shown), small holes (102) need not be present. FIG. 1 (110) shows the stent in the normal deployed configuration, pressed up against the inner wall (112) of an artery, vein, or other body lumen.

Thus when properly configured to fit into an artery or vein, the stent presses the upconverting material next to the walls of the body lumen.

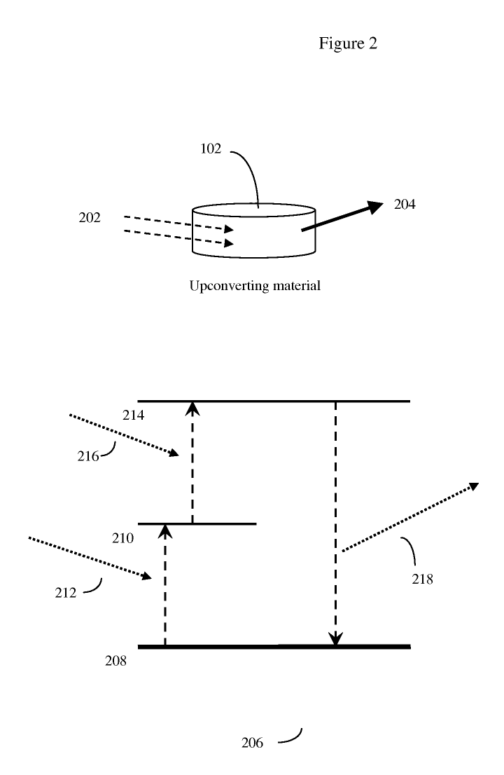

FIG. 2 shows a detail of the small disks or particles of upconverting material that may be placed into the small holes (102) or (in a finer particle form) embedded in a polymer coating the stent.

As previously discussed, on a quantum level usually the upconverting material will multiple (two or more) low energy photons (i.e. infrared light) (202), and emit a high energy upconverted photon (often an Ultraviolet light photon) (204) as a result. On a macroscopic level, of course, the effect is essentially continuous, and the upconverting disk or particle will appear to glow at a higher wavelength when illuminated with lower wavelength incident light.

The energy level diagram (206) shows the same thing from a quantum mechanics standpoint. Here an electron at a lower energy level (208) is promoted to an intermediate energy level (210) by a first long wavelength photon (212). The electron is then promoted to a higher energy level (214) by a second long wavelength photon (216). The electron then decays back to the original energy level (208) emitting a high energy short wavelength photon (218).

A large number of different upconverting materials have been identified, and the list provided later in this document is by no means exhaustive. In general, the upconverting material should be selected to ideally accept photons that can travel a fair distance through the body (in a preferred mode, in the 600 to roughly 800 nm region), upconvert the light (with as high a quantum efficiency as feasible) and emit the light at a shorter wavelength designed to be suitable for the therapeutic application contemplated. Often this emitted light will be at a wavelength of 500 nm or less. The upconverting material will usually be designed to be sable at body temperatures, and being capable of being at least embedded in a carrier or support that is non-toxic and biocompatible enough as to be suited for that particular medical application.

### Examples of Upconverting Materials:

Paz-Pujalt et. al, U.S. Pat. No. 5,786,102, incorporated herein by reference, taught a amorphous upconversion phosphor comprising barium fluoride a combination of rare-earth fluorides including yttrium and lanthanum and dopants, and a waveguide thereof on a substrate selected to have a refractive index lower than a thin film of the phosphor material or any other substrate with an appropriate buffer layer of lower refractive index that the film wherein infrared radiation and visible light are converted to ultraviolet and visible light. The amorphous upconversion phosphor is deposited at temperatures low enough to permit integration into semiconductor materials.

Chen, in U.S. Pat. No. 7,008,559, incorporated herein by reference, taught manganese doped upconversion luminescent nanoparticles.

Daqin Chen, Yuansheng Wang, Yunlong Yu, Feng Liu, and Ping Huang, “Sensitized thulium ultraviolet upconversion luminescence in Tm³⁺/Yb³⁺/Nd³⁺ triply doped nanoglass ceramics,” Opt. Lett. 32, 3068-3070 (2007) describe intense four- and five-photon ultraviolet upconversion processes through sensitization of Tm3+ ions in transparent SiO2-Al2O3-NaF-YF3 glass ceramics triply doped with Tm3+/Yb3+/Nd3+ under 796 nm excitation were investigated. Judd-Ofelt analyses evidenced the incorporation of rare-earth ions into the precipitated β-YF3 nanocrystals. In contrast with the triply doped one, no ultraviolet upconversion luminescence was observed in the Tm3+/Nd3+ codoped glass ceramic, indicating that Yb3+ acts as bridging ions to enhance the energy transfer efficiency between Nd3+ and Tm3+.

Daqin Chen, Yuansheng Wang, Yunlong Yu, and Ping Huang Intense ultraviolet upconversion luminescence from Tm3+/Yb3+:beta-YF3 nanocrystals embedded glass ceramic Appl. Phys. Lett. 91, 051920 (2007); doi:10.1063/1.2767988.

Chen found that the infrared to ultraviolet upconversion emissions of Tm3+1I6-->3F4 (346 nm) and 1D2-->3H6 (362 nm) transitions, originating from the five- and four-photon upconversion processes, respectively, were observed in the Tm3+/Yb3+ codoped precursor glass and glass ceramic containing beta-YF3 nanocrystals. The ultraviolet luminescence of the glass ceramic is 30 times stronger than that of the precursor glass, which could be attributed to the decreased probability of the 3F2-->3F4 transition and the increased cross relaxation of 3F2+3H4-->3H6+1D2 resulted from the partition of rare earth ions into nanocrystals.

### Light Sources:

As previously discussed, in the case of a stent, or other medical implant, the IR to UV upconverting material can often be illuminated by a IR light source from outside of the artery, or even from some distance away. If the light source is sufficiently powerful and at an optimized wavelength, the illumination light may even come from outside of the body.

The reason why the IR light source can be located some distance away from the upconverting stent or other medical device containing upconverting material is that the main light absorbing molecule in the human body is hemoglobin, and hemoglobin has very low absorption in the IR region.

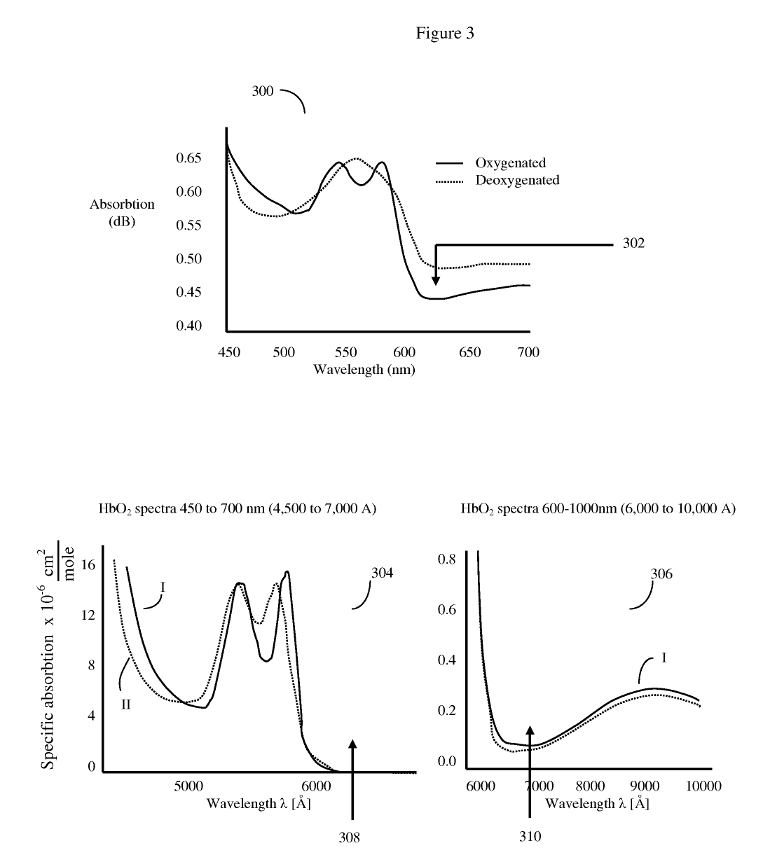

The spectrum of oxyhemoglobin is shown in FIG. 3.

Graph (300) is taken from the hemoglobin absorption spectra of Lang Quin Liu, et. al., Journal of Rehabilitation Research and Development, 43(4), July/August 2006, pages 553-564. This shows the spectra of oxy and deoxy hemoglobin in the 450 to 700 nm region. As is shown by (302), illumination light below about 600 nm penetrates far into the body because it is not absorbed by hemoglobin (the main light absorbing component in the body).

Graphs (304) and (306) are taken from Horecker, “The absorption spectra of Hemoglobin and its derivatives in the Visible and Near Infra-Red Regions”, Journal of Biological Chemistry, 1943, page 177. (304) shows the oxyhemoglobin spectra from about 425 nm to about 700 nm, and (306) shows the spectra from about 800 nm to 1000 nm. (Due to the age of the graph, the actual units are in Angstroms, where 10 angstroms=1 nm).

As (308) and (310) also show, illumination light in 600-800 nm regions (or alternatively the 650-800 nm region to avoid the region between 600 and 650 where residual tissue absorbtion is still present) penetrates far into the body because it is not absorbed by hemoglobin (the main light absorbing component in the body). Although the 600-800 nm region will thus be given in this disclosure as an example of a potentially preferred set of wavelengths useful for deep body penetration, it should be understood that this example is not intended to be limiting, and indeed inspection of FIG. 3 will show that wavelengths longer than 800 nm may also be used, because these wavelengths still penetrate far further into tissue than wavelengths less than 600 nm.

As a result, as previously discussed the IR illumination light source may be located outside the heart and potentially even outside the body. The IR light source will penetrate past the near-transparent hemoglobin, and hit the upconverting material on the sides of the stent. The upconverting material will then emit UV light.

For example, Vivian Heyward and Dale Wagner, in “Applied body composition assessment”, 2^{nd} edition (2004), page 100 teach that Near Infrared light can penetrate the tissues for distances of up to 4 centimeters. Certain equations useful for calculating light penetration in tissue as a function of wavelength are given by Khalil et. al. in U.S. Pat. No. 7,043,287, the contents of which are incorporated herein by reference. Similarly in Ella, in US patent application 20070027411 [0014] teach that near infrared Low Level Laser Treatment light penetrates the body to a depth between 3-5 cm.

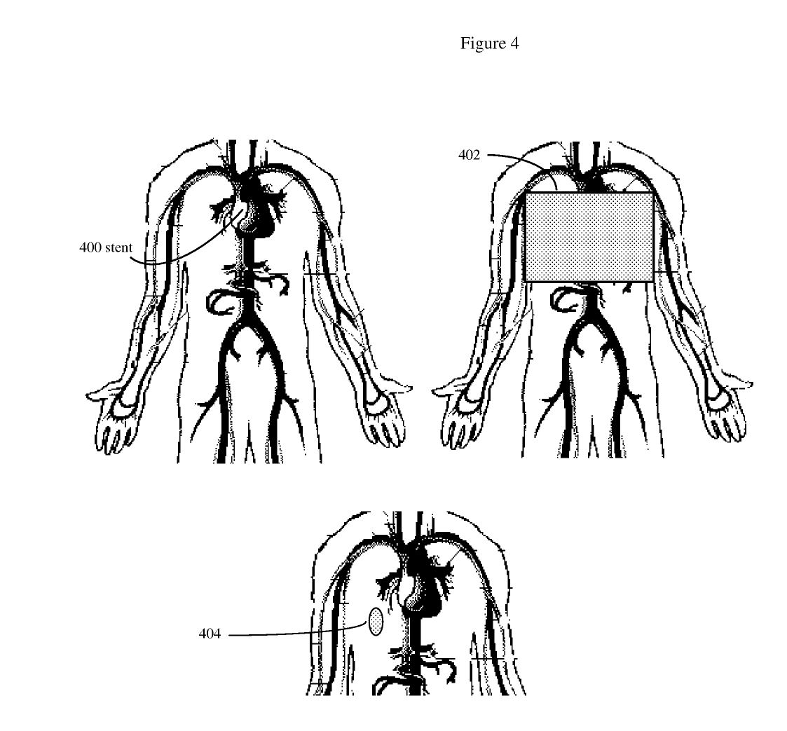

A diagram of the heart and body, showing various placement possibilities for the illumination light source, is shown in FIG. 4.

FIG. 4 (400) shows showing an upconverting stent implanted in a coronary artery. Here FIG. 4 (402) shows an example of an IR light source outside of the body—For example, a body wrap with a large (hundreds or thousands) number of IR LED diodes. Alternatively other light sources, such as one or more infrared lasers, may also be used.

FIG. 4 (404) shows an alternative example, where, the IR light source could be applied from inside the body, either through an endoscope, alternatively through actually implanting IR light sources (e.g. IR LED's or fiber optics), or other means.

One way to do this is to produce a medical implant consisting of one or more infrared diodes or infrared lasers near the site of interest, potentially run an induction coil or other wireless means of powering the device to near the surface of the body, for example under the skin, and then power the infrared medical implant by supplying power to the induction coil. Here wireless power is applied to the induction coil or other wireless power receiver implanted under the patient's skin. Wires from the receiver transmit power to the infrared light producing implant, and the implant in turn supplies infrared light to the upconverting implant, typically located within 4-5 cm or less from the infrared light producing implant.

Given that the depth of infrared penetration into tissue is about 4-5 cm, other schemes to administer infrared light may also be used. This may include temporary insertion of optical catheters or endoscopes, or other means.

For indwelling catheters, depth of infrared light penetration will typically be less of a problem because many infections occur close to the surface of the body, less than 4-5 cm deep.

As previously discussed, the UV light may be cytotoxic in itself (i.e. kill unwanted cells that are proliferating into the stent), or alternatively the cytotoxic effects of the UV light may be enhanced by administering various types of UV activated cytotoxic drugs, such as psorlens.

Use of shorter wavelength (blue or UV light) activated drugs and photosensitized agents.

The cytotoxic effects of the UV emitting stent could be enhanced by administering UV activated cytotoxic drugs, or other photosensitized agents. Examples of potentially useful drugs and agents are shown below:

High solubility psoralen compounds were taught by Hearst et. al., U.S. Pat. No. 4,169,204. Psorlens kill cells by cross-linking DNA strands when the psorlens are illuminated by UV light.

Bisaccia et. al., in U.S. Pat. No. 5,284,869, the contents of which are incorporated herein by reference, taught that the occurrence of restenosis following percutaneous transluminal coronary angioplasty is prevented or inhibited using a photopheresis treatment method. In accordance with the photopheresis treatment method, a photoactive compound such as 8-methoxypsoralen is administered to the patient's blood or affected tissue, or some fraction thereof, in vitro or in vivo using conventional administration routes. A portion of the patient's blood or affected tissue is then treated (preferably, extracorporeally) using photopheresis, which comprises subjecting the blood or affected tissue to electromagnetic radiation in a wavelength suitable for activating the photoactive compound, such as ultraviolet light, preferably long wavelength ultraviolet light in the wavelength range of 320 to 400 nm, commonly called UVA light. The treated blood or affected tissue, or a fraction thereof, is returned to the patient (in the case of extracoporeal photopheresis) or remains in the patient (following in vivo photopheresis).

Van Tassel et. al. in U.S. Pat. No. 6,719,778 taught that photoactivatable agents can also be used such that the energy activates the photoactivatable agent to cause a thickening of the vessel wall. For example, ultra-violet radiation can be used alone or in conjunction with a photoactivatable agent, such as a psoralen compound, to increase the adventitial volume of a blood vessel. Upon exposure to radiation, preferably ultra-violet-A radiation, the photoactivatable agent becomes activated and causes compositional and/or structural changes in the adventitia. However most stent problems are caused by unwanted tissue hyperplasia occurring in the stent area.

Various photoactivatable compounds are taught by Robinson et. al. U.S. Pat. No. 6,008,211, the contents of which are incorporated herein by reference.

Robinson teaches a broad class of photosensitive compounds having enhanced in vivo target tissue selectivity and versatility in photodynamic therapy. Many furocoumarin compounds, such as psoralens, exhibit cytostatic activity when photoactivated but exhibit little in vivo specificity for selectively accumulating in any particular target tissue such as atheromatous plaques. Reactive Oxygen Producing Photosensitizers (“ROPPs”) are photoactivatable compounds having an affinity for hyperproliferating cells (such as atheromatous plaque cells), which when photoactivated, produce cytotoxic reaction products. The photoactivity of a ROPP, such as a porphyrin, may be reduced by metalating the porphyrin while the selective affinity of the metalized ROPP for hyperproliferating tissue remains substantially unchanged. By linking a furocoumarin compound to a ROPP to form a F-ROPP, the cytostatic properties of the furocoumarin portion of the F-ROPP can be exploited while the selective affinity of the ROPP portion of the compound for hyperproliferating cells such as atheromatous plaque provides enhanced tissue selectivity without cytotoxicity. In vivo, certain F-ROPPs may be forced to selectively accumulate in a target tissue by illuminating only the target tissue with light having a wavelength operable for photoactivating the F portion of the F-ROPP thereby causing the F-ROPP to either form a monoadduct with or crosslink the cellular DNA in the target tissue. Light of a second wavelength can then be delivered to the target tissue to photoactivate the ROPP portion causing further interference with cellular activity.

Robinson's table of potentially useful UV activated drugs (his Table I) includes Pyrrole-derived macrocyclic Texaphyrins and derivatives compounds thereof (11) Naturally occurring or synthetic Phenoxazine dyes and derivatives porphyrins and derivatives thereof (12) thereof (1)* Phenothiazines and derivatives Naturally occurring or synthetic thereof (13) chlorins and derivatives thereof (2) Chalcoorganapyrylium dyes and Naturally occurring or synthetic derivatives thereof (14) bacteriochlorins and derivatives Triarylmethanes and derivatives thereof (3) thereof (15) Synthetic isobacteriochlorins and Rhodamines and derivatives thereof (4) thereof (16) Phthalocyanines and derivatives Fluorescenes and derivatives thereof (5) thereof (17) Naphthalocyanines and derivatives Azaporphyrins and derivatives thereof (6) thereof (18) Porphycenes and derivatives Benzochlorins and derivatives thereof (7) thereof (19) Porphycyanines and derivatives Purpurins and derivatives thereof (8) thereof (20) Pentaphyrin and derivatives Chlorophylls and derivatives thereof (9) thereof (21) Sapphyrins and derivatives Verdins and derivatives thereof (22) thereof (10)

Other compositions were also taught by Robinson et. al., U.S. Pat. No. 5,773,609, incorporated herein by reference.

Gantz, in U.S. Pat. No. 6,764,501, incorporated herein by reference, taught that atherosclerotic vascular disease can be treated by exposing the interior of an artery or vein to visible or ultraviolet light of a selected spectrum to thereby sterilize the artery, eradicating infectious pathogens and reverse the pathogenesis of atherosclerotic artery disease. A fiber optic bundle positioned via an intra-arterial catheter is connected to an appropriate light radiation source located outside of the body during treatment. In another form, the light energy is produced by means of a light energy source located at the distal tip of the instrument shaft positioned centrally within the atherosclerotic plaque formation. In this case, the power source at the distal tip of the instrument can be a light emitting diode (LED) or a transparent tube containing a chemilumlumenescent substance for producing cool light energy within the vessel to destroy the pathogenic microorganisms in the surrounding plaque or vessel without damage to the body tissue of the patient.

Here, the methods differ substantially from those of Gantz because no fiber optic bundle is being placed in the interior of an artery or vein.

Other photosensitized agents may also be used. For example, various forms of Titanium Dioxide (TiO₂) can be photoactivated to a form that can kill microorganisms (i.e. bacteria, fungi). Although previously, use of such photoactivated antimicrobial agents for some applications was hampered by the difficulty of providing short wavelength photoactivation light to medical devices implanted in the body, such as catheters, use of upconverting materials solves this problem. Medical devices employing photoactivated antimicrobial agents may be constructed using upconverting materials, and then illuminated with longer wavelength light, such as light in the 600-800 nm region, that penetrates the body well. The upconverting material on the medical device will convert the longer wavelength light into shorter wavelength light capable of photoactivating the photoactivated anti-microbial agent. The photoactivated anti-microbial agent can, in turn, destroy bacteria, fungi, or other microbes near the implanted medical device.

Alternatively the photoactivated agent may be used to exert a cytotoxic effect against the patient's cells as well. For example, a stent composed of a mixture of an upconverting material and photoactivated agent (again using TiO2 as an example) will destroy unwanted human cells, such as proliferating cells causing unwanted restenosis, by cytotoxic chemicals, such as free radicals, produced by the photoactivated agent.

### Drug Use Examples:

In one treatment mode, a patient is administered UV activated cytotoxic drugs, and kept away from other UV light sources. The upconverting stent is illuminated by infrared (IR) light. The stent produces UV light by the upconversion process exactly where it is needed—that is, local to the stent are where the chances of unwanted cell proliferation and hyperplasia is greatest. The UV activated cytotoxic drugs are carried to the stent area, where they interact with the UV light emitted by the stent, and kill the unwanted proliferating cells.

One advantage of this approach that the cytotoxic effect of the stent can have a very long lifetime. Unlike drug eluting stents, where the stent eventually runs out of the drug, an IR to UV upconverting stent could have a potential lifetime of years or more, because it never runs out of material. That is, the upconverting material can be made of a glass or ceramic or other long-life material, and produce UV photons whenever illuminated with outside light.

Use with Drug Pumps

In some cases, the efficiency and selectivity of drug administration may be further enhanced by delivering the light activated drug to an area of the body local or “upstream” (from a circulatory sense) from the implanted upconverting medical device. In this manner, drug selectivity is enhanced both by the short range nature of the shorter wavelength photons emitted by the implanted upconverting medical device, and by the localized nature of the drug administration pump.

### Other Concepts:

In an alternative embodiment, the stent could contain a plurality of UV light emitting diodes, and suitable energy harvesting circuitry to power the UV light emitting diodes from an external radiofrequency source.

In other alternative embodiments, holes in the stent may contain one more sensing elements. These sensing elements would be used to determine if undesired cell hyperplasia had occurred. Examples of such sensing elements can include a flexible membrane placed over one or more small holes in the stent. The flexible membrane may contain magnetic material and/or electrically conducting material. When exposed to an oscillating magnetic material, the flexible membrane will vibrate at a first set of frequencies if the membrane is not covered with overgrown cells, but will vibrate at a second set of frequencies if the membrane is not covered with the cells. This difference in vibration frequencies could be detected by suitable magnetic or radiofrequency pickups, and may be used to diagnose the status of the stent prior to treatment.

In yet another alternative environment, holes in the stents might contain magnetic fibers or filaments that can be induced to move by external magnetic fields, and be used to actively clean portions of the stent area.

It should be understood that although the stent example and the cateter example have been used as specific examples of an implantable medical device incorporating an upconverting material, other implantable medical devices may also be improved by the incorporation of upconverting material.

Examples of other applications include implantable upconverting “seeds” for treatment of malignant or non-malignant growths or cancer. Here often one or more (i.e. a plurality) of IR to UV upconverting pellets or seeds comprised of upconverting material will be implanted in the region where cell death is desired. Cell death will be mediated by exposing the pellets or seeds to IR light, often in conjunction with a light activated cytotoxic drug, photoactivated catalyst, or other light activated drug or carrier mechanism of interest.

In alternative embodiments, the light activated drug may not be directly cytotoxic, but may be light activated to a form that causes the drug to act as a signal to other drugs or drug carrier moieties, informing the moieties that cells in the region of the light activated drug should be targeted for various forms of chemical therapy or biologic therapy.

For example, upconverted photons emitted by the device could convert a first apo-target molecule from a form that does not serve as a target for another receptor molecule (such as an antibody or protein) to a form (target-molecule) that now does server as a target for the other receptor molecule. Examples of such molecules that can be converted from a first apo-target form to a second target-form include various light receptor molecules (i.e. retinal, rhodopsin, G-proteins, UV light receptor complexes, various light receptor proteins, LOV domain proteins, and the like).

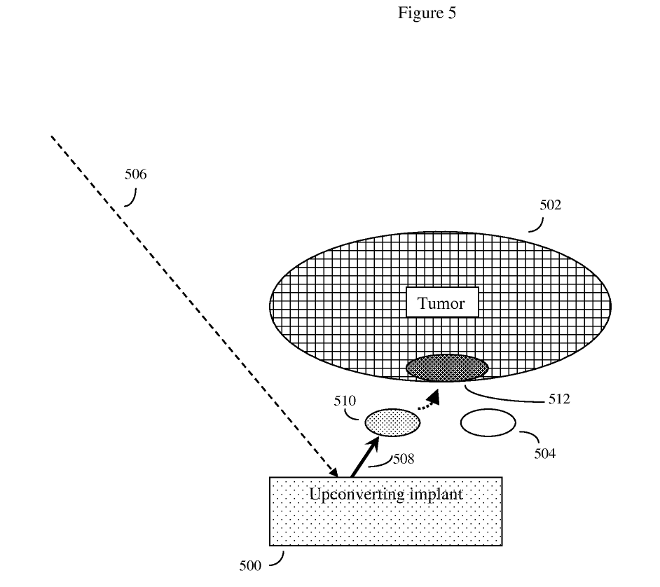

FIG. 5 shows an example of how an upconverting implant may interact with a light sensitive drug to produce a desired therapeutic treatment. In FIG. 5 (500), an upconverting implant is implanted in or near a diseased portion of the body, such as a tumor (502). The tumor (502) is treated with a light sensitive drug (504) that is inactive or less active when not exposed to short wavelength light. When exposed to longer wavelength excitation light (506), the upconverting material emits shorter wavelength light (508). This shorter wavelength light converts at least some of the light sensitive drug (504) to an active form (510). The active form of the drug, in this example, is a cytotoxic drug that in turn damages a nearby portion of the tumor (512).

In other embodiments, the implantable medical device may be comprised of small millimeter sized, fractional millimeter sized, or even microscopic (5000 microns or less) particles that can have their surface properties modified to make them water soluble, fat soluble, insoluble, or bind to specific organs or locations of the body (for example by binding specific antibodies, receptors, ligands, or targets to the particles). In this embodiment, the particles may be administered into the blood circulation as a dye, injected into organs, swallowed and allowed to enter the gastrointestinal tract, and the like, and used to visualize the status of various organs. Often this will be done in conjunction with another dye system that can detect the shorter wavelength photons emitted by the upconverting material, and in turn convert this to a longer wavelength signal that has sufficient tissue penetrating power to exit the body and be detected by detectors outside of the body. In other cases, the detectors may be mounted on endoscopes, and the like, and introduced into the body. In these cases, the shorter wavelength signal may be detected directly.

In this situation, use of small or microscopic particles of upconverting material may also be used for therapeutic applications. For example, upconverting particles may be introduced into the circulation with a modified surface rendering the particles capable of binding to cells (i.e. immune system cells) or tissues of interest, and these particles, in conjunction with light sensitive drugs, may also effect therapeutic treatment of the desired cells or tissues.

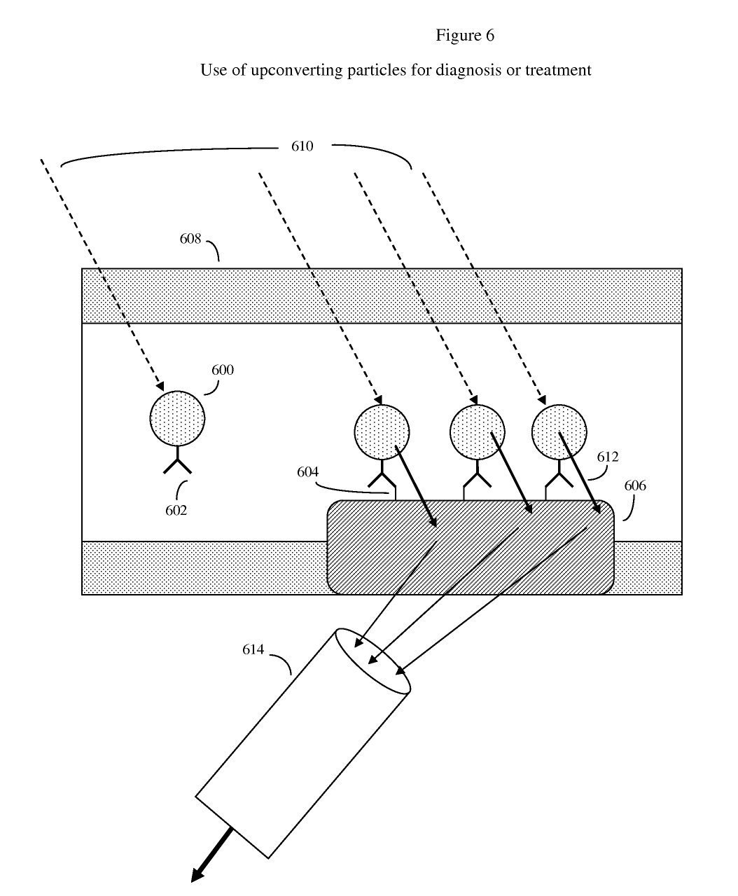

FIG. 6 shows an example of how small (sub millimeter) soluble particles of upconverting material might be used for a medical diagnostic procedure. In this example, the soluble particles (600) are first bound to an antibody (602) against a molecular target (604) on a tissue or cell (often a diseased tissue or cell) of interest (606). The particles are then introduced into a body lumen (608), which could be into the blood stream, into the gastrointestinal tract, into the lung, into urinary tract, or other area. The upconverting particles will tend to preferentially bind to the tissue or cells that express the target (606).

In this diagram, the body lumen is an artery or vein, and the upconverting particles are being carried along by the flow of the blood circulation.

When illuminated with longer wavelength light (610), the upconverting particles will produce shorter wavelength light (612), that is concentrated over the target of interest. For a diagnostic application, this shorter wavelength light may be observed directly, in this example by an optical endoscope (614), or by other means. Alternatively the upconverting particles may be used to therapeutically treat the tissue or cells either directly by delivering cytotoxic UV light, or indirectly by way of the light sensitive drug systems discussed previously.

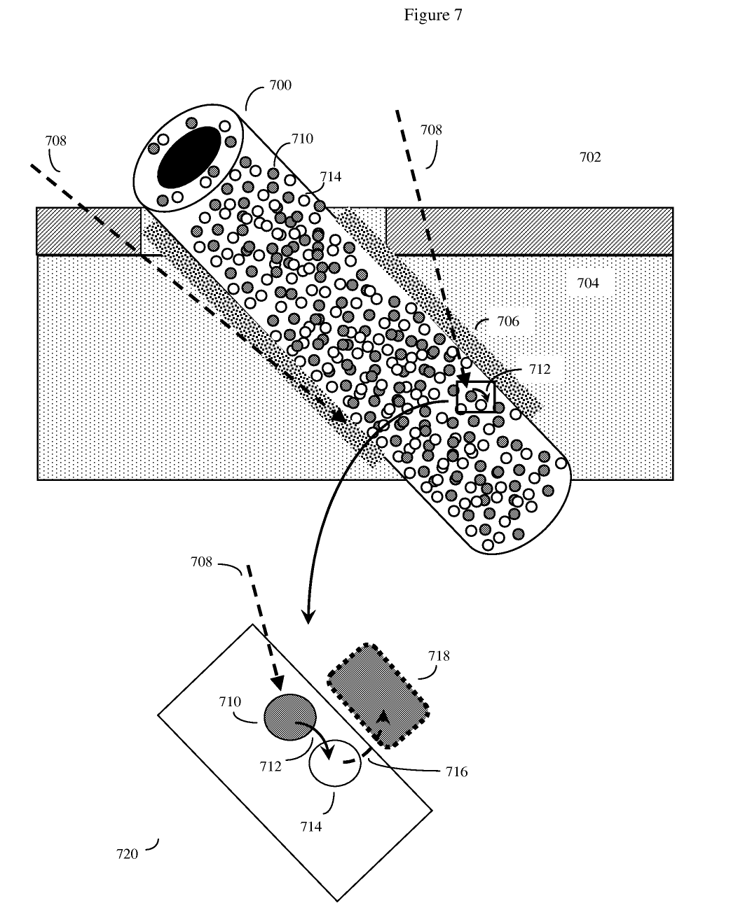

FIG. 7 shows a medical catheter, such as a Broviac® catheter, or other catheter, that spans the space between outside of the patient's body and inside the patient's body, and thus poses a considerable risk of contamination by various microorganisms at the interface, and subsequent infection.

Here, risk of infection can be reduced by constructing the catheter with upconverting particles, as well as particles of a photocatalytic antibacterial material, such as particles of titanium dioxide on or near the surface of the catheter. Use of other agents, such as silver (Ag), may also be added to improve the anti-microbial effect.

FIG. 7 (700) shows such a catheter, spanning the region from outside the patient's body (702) to inside the patient's body (704). In this example, there is an infection at the junction, and thus the catheter (700) is surrounded by a biofilm (706) undesired microbes such as pathogenic bacteria.

These microbes are destroyed by illuminating the catheter with 600-800 nm light (708), which penetrate deeply into the body. The upconverting particles (710) produce shorter wavelength light (712), which activates the photoactivated antibacterial material (714). The photoactivated antibacterial material in turn kills (716) the various undesired pathogenic bacteria in the biofilm (718). A higher magnification view of this process is shown in (720).

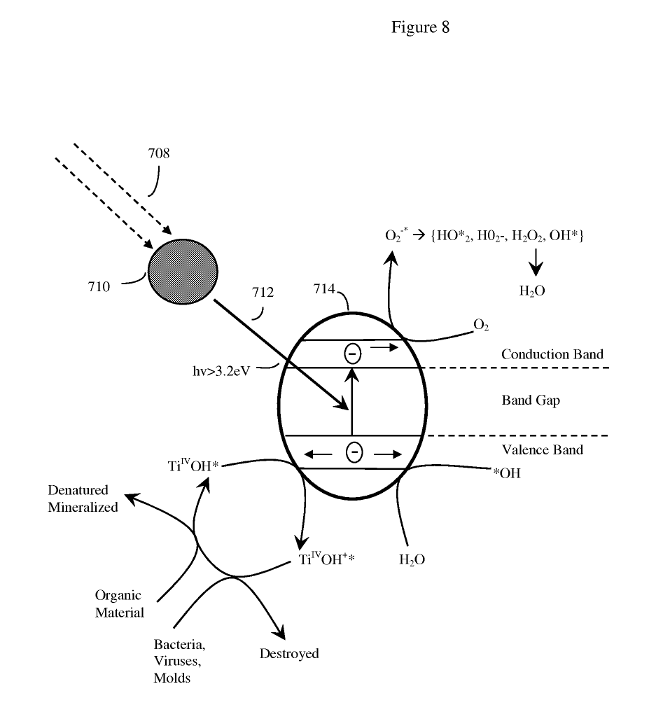

Various types of photocatalytic antimicrobial materials may be used. These materials include Na-Pheophorbide, semiconductor particles, particles such as Titanium Dioxide (TiO₂), as well as various forms of TiO₂ such as nitrogen- and carbon-doped TiO2 substrates, and other suitable photoactivated antimicrobial or cytotoxic agents. Such agents may include various doped and non-doped forms of TiO₂, NaTaO₃, ZnO, CdS, GaP, SiC, WO₃, ZnS, CdSe, SrTiO₃, CaTiO₃, KTaO₃, Ta2O₅ ZrO₂, and so on. In some embodiments, it may be useful to enhance the antimicrobial activity of the upconverting medical device by also using other agents, such as Ag (silver) nanoparticles, to increase the efficiency of the antimicrobial or cytotoxic process. One proposed mechanism for this cytotoxic (antimicrobial) effect, taken from FIG. 15 of Francis (US patent application 2008/0039768) is shown in FIG. 8. FIG. 8 shows a detail of some of the reactions that may be going on inside of the photoactivated catalyst (714) previously shown in FIG. 7. As before, longer wavelength infrared light (708) is absorbed by upconverting particle or material (710), which emits a shorter wavelength light (712) which in turn is absorbed by, and activates, photoactivated catalyst (714).

Although in some embodiments, particles of upconverting material and particles of a photoactivated antimicrobial agent (or particles of a cytotoxic agent) may be a mix individual particles of the various agents in close proximity, in other embodiments, it may be useful to fabricate hybrid particles comprised of the upconverting material and the photoactivated cytotoxic agent or agents in the same particle.

This approach offers a number of advantages over prior art approaches. For example, Francis, in US patent application 2008/0039768, incorporated herein by reference, taught a medical device such as a catheter with a photocatalytic layer with an antimicrobial material, such a doped semiconductor oxide (e.g. TiO₂ and other compounds), which could be illuminated by electromagnetic radiation. However because Francis could not determine how to easily send activating light to his photocatalytic compounds, he had to devise complex illumination methods such as fiber optic cables, wave guides, light ports, UV transmissible wave guides, LEDs, antenna, and the like to convey the photoactivating light to the surface of the catheter. By contrast, by also including upconverting materials into the catheter, the catheter may be simply illuminated by infrared light, in particular 600-800 nm light which penetrates deeply into tissues, from an outside source. Thus the catheter design itself may be simplified, because the light source can now be entirely outside the body if this is desired.

Note that this same approach may be used for other devices, such as stents. That is, the activated semiconducting material has a general destructive effect on organic material. Although this destructive effect may be utilized to destroy bacteria, viruses, and molds, it may also be used to destroy unwanted tissue, such as unwanted tissue acting to close an implanted stent. Thus by making medical implants, such as stents, out of a combination of an upconverting material and a photoactivated catalyst, such as TiO2, other semiconductor material (previously discussed) or other reusable photoactivated catalyst, the implant (such as the stent) may be implanted, and then used for years, retaining it's cytotoxic capability whenever illuminated with sufficient quantities of infrared light.

### Other Amplification Methods:

For some applications, it may be desirable to use naturally occurring protease cascades (e.g. coagulation protease cascades), naturally occurring inflammation pathways, cellular mediated immune responses, or cellular mediated inflammatory or phagocytic responses to amplify the relatively small number of higher energy (e.g. UV) photons that will be emitted by the upconverting implanted device or particles.

For example, ultraviolet activated haptens are known, which, upon exposure to ultraviolet light, can bind to nearby molecules, such as cell surface molecules on diseased tissue such as tumor or atherosclerotic tissue. These haptens can then signal immune system cells, such as T-cells, macrophages and the like to attack the tissue and attempt to remove it from the body.

Similarly a modified form of a protease might be used, where the protease would be photoactivated from an inactive form to an active form, and then stimulate a protease cascade directed against a nearby target.

1. An implantable medical device comprising upconverting material and a photocatalytic material, wherein when said upconverting material is exposed to red or infrared light, said upconverting material emits shorter wavelength light which is subsequently absorbed by said photocatalytic material, which in turn emits cytotoxic chemicals in response to said shorter wavelength light.

2. The implantable medical device of claim 1, in which the device is selected from the group consisting of stents, orthopedic implants, pellets, injectable semi-solid implants, suspended particles, aerosols, soluble particles, drug administration devices, coils, shunts, electrodes, needles, and catheters.

3. The device of claim 1, in which the upconverting material absorbs infrared light, and emits visible or ultraviolet light.

4. The device of claim 3, in which the infrared light comprises light of wavelengths longer than 600 nm.

5. The device of claim 3, in which the visible or ultraviolet light comprises light of wavelengths shorter than 500 nm.

6. The device of claim 1, in which the device comprises a support material comprised of a non-upconverting material, and the upconverting material is embedded in the non-upconverting material.

7. The device of claim 1, in which the photocatalytic material is a semiconductor.

8. The device of claim 1, in which the photocatalytic material is titanium dioxide.

9. An implantable medical device comprising upconverting material and photocatalytic material, in which the device comprises a support material comprised of a non-upconverting material and non-photocatalytic material, and the upconverting material and photocatalytic material is embedded in the non-upconverting material.

10. The implantable medical device of claim 9, in which the device is selected from the group consisting of stents, orthopedic implants, pellets, injectable semi-solid implants, suspended particles, aerosols, soluble particles, drug administration devices, coils, shunts, electrodes, needles, and catheters.

11. The device of claim 9, in which the upconverting material absorbs infrared light, and emits visible or ultraviolet light, and the photocatalytic material is activated by said visible or ultraviolet light.

12. The device of claim 9, in which the infrared light comprises light of wavelengths longer than 600 nm.

13. The device of claim 9, in which the visible or ultraviolet light comprises light of wavelengths shorter than 500 nm.

14. The device of claim 9, in which the device, when illuminated by photons at a first longer wavelength emits photons at a second shorter wavelength, and in which the photons at said second shorter wavelength activate the photocatalytic material, and the photocatalytic material has direct cytotoxic effects against human cells or microbes in the immediate vicinity of said device.

15. The device of claim 9, in which the photocatalytic material is a semiconductor.

16. The device of claim 15, in which the photocatalytic material is titanium dioxide.

17. The device of claim 9, used in an infrared light producing medical implant, comprising:
An infrared light source, implanted in close proximity to at least one upconverting medical implant;
A wireless power receiver, implanted under the patient's skin, and connected to the infrared light source by one or more power conducting wires or leads,
Wherein a wireless power source located outside of the patient's body supplies power to the wireless power receiver, and the wireless power receiver sends power to the infrared light source;
And said infrared light source provides infrared light to the at least one upconverting medical implant.

18. The device of claim 9, used in an infrared light conducting medical implant, comprising:
one or more optical fibers, one end of said fibers implanted in close proximity to at least one upconverting medical implant, and the other end terminating to a region less than 5 cm within the skin or interior or exterior surface of a patient,
wherein infrared light from a source located exterior to the patient, or exterior to the exterior surface of a patient supplies infrared light to the at least one optical fiber, and said at least one optical fibers convey infrared light to the upconverting medical implant.
